(19) United States
(12) Patent Application Publication
(10) Pub. No.: US 2006/0035887 A1
(43) Pub. Date: Feb. 16, 2006

(54) Process for preparing olanzapine

(75) Inventors: Sundaram Venkataraman, Hyderabad (IN); Srinivasan Thirumalai Rajan, Hyderabad (IN); Veera Venkata Naga Chandra Sekhar Bulusu, Hyderabad (IN); Ravi Kumar Kasturi, Hyderabad (IN); Suneel Kumar Kapabalu, Kakinada (IN); Kavitha Gokavalasa, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignee: DR. REDDY'S LABORATORIES LIMITED

(21) Appl. No.: 11/171,093

(22) Filed: Jun. 30, 2005

Related U.S. Application Data
(60) Provisional application No. 60/585,198, filed on Jul. 2, 2004.

(51) Int. Cl. A61K 31/551; C07D 498/02

(52) U.S. Cl. 514/220; 540/560

(57) ABSTRACT

A process for preparing olanzapine comprising methylation of N-demethyl olanzapine with a methylating agent in a solvent comprising dichloromethane, methanol, or a mixture thereof.

### CROSS-REFERENCE TO RELATED APPLICATION

This application is a nonprovisional filing of copending U.S. Provisional Application No. 60/585,198 filed on Jul. 2, 2004, the entire contents of which are incorporated by this reference.

### INTRODUCTION TO INVENTION

The present invention relates to a process for the preparation of 2-methyl-4-(4-methyl-1-piperazinyl)-10H-thieno[2,3-b][1,5]benzodiazepine (hereinafter referred to by the adopted name “olanzapine”).

Olanzapine is psychotropic agent that belongs to the benzodiazepine class, and products containing the drug are available commercially as ZYPREXA™. ZYPREXA™ is believed to work by balancing the chemicals naturally found in the brain.

EP 0454436 describes olanzapine and a process for its preparation. The process for the preparation of olanzapine comprises reacting N-methylpiperazine with 4-amino-2-methyl-10H-thieno[2,3-b][1,5]benzodiazepine hydrochloride in a mixture of dimethyl sulfoxide and toluene as a solvent medium under a nitrogen atmosphere for 20 hours. The product was filtered and crystallized from acetonitrile.

International Published Application No. WO 04/000847 discloses the process for the preparation of olanzapine. The process for the preparation of olanzapine comprises reacting 2-methyl-4-(1-piperazinyl)-10H-thieno[2,3-b][1,5]benzodiazepine (N-demethyl olanzapine), anhydrous sodium acetate, glacial acetic acid, aqueous formalin and distilled water, cooled to 0° C. followed by subsequent addition of sodium borohydride in small portions by maintaining the pH at 9 with 2N aqueous NaOH yielded olanzapine.

Alternatively, olanzapine is prepared by reacting 2-methyl-4-(1-piperazinyl)-10H-thieno[2,3-b][1,5]benzodiazepine (N-demethyl olanzapine) with methyl iodide, potassium carbonate in the presence of methanol.

Alternatively, olanzapine can also be prepared by reacting 2-methyl-4-(1-piperazinyl)-10H-thieno[2,3-b][1,5]benzodiazepine (N-demethyl olanzapine) with formic acid in presence of aqueous formaldehyde. Further, olanzapine can also be prepared by reacting 2-methyl-4-(1-piperazinyl)-10H-thieno[2,3-b][1,5]benzodiazepine (N-demethyl olanzapine) with ethyl formate in the presence of tetrahydrofuran (THF).

The processes described for the preparation of olanzapine have disadvantages for their performance in large scale as they involve some hazardous and costly chemicals like methyl iodide, which are unsafe and difficult to handle in a commercial scale. The usage of formamide as a solvent is another difficulty in the process as the recovery and reuse of said solvent is not feasible in scale up. Hence, the processes have high cost and are not suitable for commercial production. Further, the yields of olanzapine are not high.

### SUMMARY OF THE INVENTION

In one aspect, the present invention provides an improved process for the preparation of olanzapine. The process for the preparation of olanzapine of Formula 1 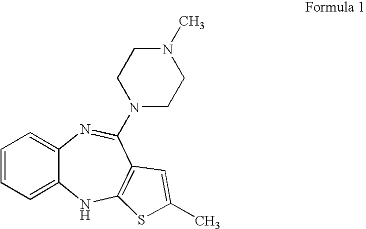
comprises methylation of 2-methyl-4-(1-piperazinyl)-10H-thieno[2,3-b][1,5]benzodiazepine (N-demethyl olanzapine) of Formula 2 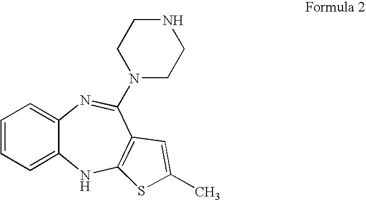
with a methylating agent, in a solvent comprising dichloromethane, methanol, or a mixture thereof.

An aspect of the invention is a process for preparing olanzapine, comprising methylation of N-demethyl olanzapine with a methylating agent in a solvent comprising dichloromethane.

Another aspect of the invention is a process for preparing olanzapine, comprising methylation of N-demethyl olanzapine with a methylating agent in a solvent comprising methanol.

A further aspect of the invention is a process for preparing olanzapine, comprising methylation of N-demethyl olanzapine with a methylating agent in a solvent comprising dichloromethane, methanol, or a mixture of dichloromethane and methanol.

### DETAILED DESCRIPTION

The present invention provides an improved process for the preparation of olanzapine.

The present inventors have surprisingly found that use of dichloromethane, methanol, or a mixture thereof as a solvent and further subjecting the product thus obtained to a further step of purification results in highly pure olanzapine.

A process for the preparation of olanzapine of Formula 1 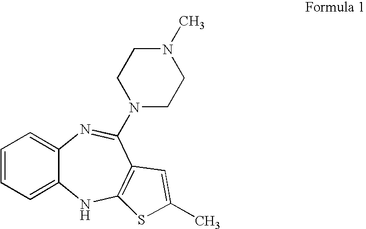
comprises methylation of 2-methyl-4-(1-piperazinyl)-10H-thieno[2,3-b][1,5]benzodiazepine (N-demethyl olanzapine) of Formula 2 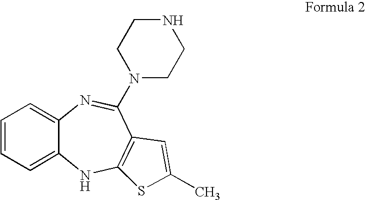
with a methylating agent in the presence of a base, in a solvent comprising dichloromethane, methanol, or a mixture thereof, and subjecting the product thus obtained to desired further purification.

Examples of useful methylating agents include dimethyl sulfate, mixtures of formaldehyde and formic acid, and methyl iodide. Dimethyl sulfate has been found particularly useful as a methylaing agent.

Methylation is carried out in a solvent comprising dichloromethane and/or methanol, in the presence of a base such as sodium hydroxide, sodium carbonate, sodium bicarbonate, sodium methoxide, potassium hydroxide, potassium carbonate, potassium bicarbonate, potassium tertiary-butoxide or mixtures thereof, preferably sodium hydroxide, dissolved in a solvent such as a C₁-C₅ alcohol such as methanol, ethanol, 2-propanol, 2-butanol, tertiary-butyl alcohol, pentanol or any mixture thereof, preferably methanol, below about 35° C., such as about −20 to 10° C, or about −15 to 5° C., or about −10 to 0° C., followed by isolation of crude olanzapine.

The crude olanzapine thus obtained can be subjected to further desired steps of purification. A step of purification can comprise dissolving the resulting crude product in a solvent such as N,N-dimethyl formamide, 1,4-dioxane, acetonitrile, acetone, or any mixture thereof, preferably N,N-dimethyl formamide, followed by addition of water to precipitate out the compound and then separating the pure olanzapine.

Alternatively, crude olanzapine can be purified by dissolving in a solvent such as dimethyl sulfoxide and adding an inorganic or organic acid such as hydrochloric acid, hydroiodic acid, acetic acid, or sulfuric acid, preferably acetic acid, followed by adding an antisolvent such as water, n-hexane, n-heptane, or any mixture thereof, preferably water, and separating the pure olanzapine.

Accordingly, an exemplary embodiment of the process for the preparation of olanzapine comprises:
i. reacting 4-amino-2-methyl-10H-thieno-[2,3-b][1,5]benzodiazepine hydrochloride with piperazine in a solvent such as: N,N-dimethyl formamide; a C₁-C₅ alcohol such as 2-propanol, 2-butanol, or tertiary-butyl alcohol; a glycol such as diethylene glycol; a halogenated hydrocarbon solvent such as chloroform, dichloromethane, or 1,2-dichloroethane; 1,4-dioxane; tetrahydrofuran; dimethyl sulfoxide; toluene; xylene; hexane; cyclohexane; n-heptane; or a mixture of any two or more thereof, preferably a mixture of dimethyl sulfoxide and toluene, and stirring the mixture at temperatures up to reflux conditions until the reaction is complete;
ii. quenching the reaction solution of step (i) with water and subsequent isolation of a crude compound;
iii. dissolving the compound of step (ii) in an aqueous organic acid such as formic acid or acetic acid, preferably acetic acid, washing the resulting solution with a water immiscible solvent such as toluene, a halogented hydrocarbon solvent such as chloroform or dichloromethane, or ethyl acetate, preferably dichloromethane;
iv. rendering the aqueous solution of step (iii) basic with a base such as sodium hydroxide, sodium carbonate, sodium bicarbonate, potassium hydroxide, potassium carbonate, potassium bicarbonate, or potassium tertiary butoxide, preferably sodium hydroxide, to afford the pure compound 2-methyl-4-(1-piperazinyl)-10H-thieno[2,3-b][1,5]benzodiazepine (demethyl olanzapine);
v. subsequent methylation of the compound of step (iv) with a methylating agent such as dimethyl sulfate, a mixture of formaldehyde and formic acid, or methyl iodide, preferably dimethyl sulfate; in a solvent such as N,N-dimethyl formamide, a C₁-C₅ alcohol such as 2-propanol, 2-butanol, or tertiary-butyl alcohol, a glycol such as diethylene glycol, or a halogenated hydrocarbon solvent such as chloroform, dichloromethane or 1,2-dichloro ethane, or a mixture of any two or more thereof, preferably dichloromethane, methanol, or a mixture of dichloromethane and methanol, in the presence of a base such as sodium hydroxide, sodium carbonate, sodium bicarbonate, sodium methoxide, potassium hydroxide, potassium carbonate, potassium bicarbonate, potassium tertiary butoxide, or any mixture thereof, preferably sodium hydroxide, dissolved in a solvent such as a C₁-C₅ alcohol such as methanol, ethanol, 2-propanol, 2-butanol, tertiary-butyl alcohol, pentanol, or any mixture thereof, preferably methanol, at a temperature below about 35° C., such as about −20 to 10° C., or about −15 to 5° C., or about −10 to 0° C.;
vi. quenching the reaction solution of step (v) with water;
vii. separation of the aqueous layer from the reaction mass of step (v) and extracting the product into an organic solvent such as is used in step (v);
viii. washing the combined organic phase of step (vii) with water followed by an aqueous C₁-₆ organic acid or an inorganic acid such as hydrochloric acid, preferably acetic acid,
ix. distillation of the organic solvent from the organic phase of step (viii), such as under reduced pressure;
x. dissolving the resulting crude product of step (ix) in a solvent such as N,N-dimethyl formamide, dimethyl sulfoxide, 1,4-dioxane, acetonitrile, acetone, or any mixture thereof, preferably N,N-dimethyl formamide, followed by addition of water to precipitate out the product compound; and
xi. separation of the solid compound.

The following examples illustrate certain aspects of the present invention, and are not intended to limit the scope of the invention as defined by the appended claims.

### EXAMPLE 1

### Preparation of 2-Methyl-4-(1-Piperazinyl)-10H-Thieno[2,3-b][1,5]Benzodiazepine (Demethyl Olanzapine)

A mixture of 4-amino-2-methyl-10H-thieno-[2,3-b][1,5]benzodiazepine hydrochloride (500 g), piperazine (568 g), dimethyl sulfoxide (500 ml) and toluene (2000 ml) was heated to reflux. The reaction mass was maintained at reflux for 5 hours and then cooled to ambient temperature. Water (2500 ml) was added slowly and the solid that separated was filtered and washed with toluene, followed by water. The wet compound was then dissolved in a mixture of acetic acid (216 ml) and water (2500 ml) and washed with dichloromethane (6×250 ml). The resulting solution after washing was made basic with sodium hydroxide solution (40%, 212 ml). The solid that formed was filtered, washed with water and dried to yield 2-methyl-4-(1-piperazinyl)-10H-thieno[2,3-b][1,5]benzodiazepine (Yield: 400 g).

### EXAMPLE 2

### Preparation of Olanzapine

2-methyl-4-(1-piperazinyl)-10H-thieno[2,3-b][1,5]benzodiazepine (300 g) obtained from Example 1 was charged into dichloromethane (3000 ml) with stirring and the mixture was cooled to below 0° C. Dimethyl sulfate (286 ml) was added at the same temperature slowly and the temperature was maintained. A solution of sodium hydroxide (242 g) in methanol (1500 ml) was cooled to 0-5° C. and added slowly to above reaction mass, which was maintained below 0° C. Maintenance was continued until the reaction was substantially complete. Added water (1500 ml) and separated the aqueous layer. The organic layer was washed with water followed by aqueous acetic acid. Finally after water washing of the organic phase, dichloromethane was evaporated and the resulting residue was dissolved in N,N-dimethyl formamide (400 ml). To the obtained solution, water (100 ml) was added to get pure olanzapine (84 g). Purity 99.8% by HPLC.

### EXAMPLE 3

### Preparation of Olanzapine

2-methyl-4-(1-piperazinyl)-10H-thieno[2,3-b][1,5]benzodiazepine (200 g) obtained from Example 1 was charged into methanol (2400 ml) with stirring and the mixture was cooled to below 0° C. Dimethyl sulfate (127.4 ml) was added at the same temperature slowly and the temperature condition was maintained. A solution of sodium hydroxide (107.4 g) in methanol (600 ml) was cooled to 0-5° C. and added slowly to the above reaction mass, which was below 0° C. The temperature was maintained until the reaction was substantially complete. The reaction mass was quenched with water. Separated solid was washed with water followed by methanol. The resulting crude product was purified by dissolving in dimethyl sulfoxide, followed by acetic acid addition, and then water was added to precipitate out the compound. The separated compound was filtered and washed with a mixture of dimethyl sulfoxide and water. This solid was further purified by dissolving in dimethyl sulfoxide, adding acetic acid followed by adding water to precipitate out the compound. The separated solid was filtered. This pure product was finally dissolved in dimethyl sulfoxide and sodium bicarbonate solution was added to it. The separated solid was filtered to afford pure olanzapine (80 gms). Purity 99.8 % by HPLC.

We claim:

1. A process for preparing olanzapine, comprising methylation of N-demethyl olanzapine with a methylating agent in a solvent comprising dichloromethane.

2. The process according to claim 1, wherein a solvent further comprises methanol.

3. The process according to claim 1, wherein a methylating agent comprises dimethyl sulfate, or a mixture of formaldehyde and formic acid, or methyl iodide.

4. The process according to claim 1, wherein a methylating agent comprises dimethyl sulfate.

5. The process according to claim 1, wherein methylation occurs in the presence of a base.

6. The process according to claim 1, wherein methylation occurs in the presence of a base comprising sodium hydroxide, sodium carbonate, sodium bicarbonate, sodium methoxide, potassium hydroxide, potassium carbonate, potassium bicarbonate, potassium tertiary butoxide, or a mixture of any two or more thereof.

7. The process according to claim 1, wherein methylation occurs in the presence of sodium hydroxide.

8. A process for preparing olanzapine, comprising methylation of N-demethyl olanzapine with a methylating agent in a solvent comprising methanol.

9. The process according to claim 8, wherein a methylating agent comprises dimethyl sulfate, or a mixture of formaldehyde and formic acid, or methyl iodide.

10. The process according to claim 8, wherein a methylating agent comprises dimethyl sulfate.

11. The process according to claim 8, wherein methylation occurs in the presence of a base.

12. The process according to claim 8, wherein methylation occurs in the presence of a base comprising sodium hydroxide, sodium carbonate, sodium bicarbonate, sodium methoxide, potassium hydroxide, potassium carbonate, potassium bicarbonate, potassium tertiary butoxide, or a mixture of any two or more thereof.

13. The process according to claim 8, wherein methylation occurs in the presence of sodium hydroxide.

14. A process for preparing olanzapine, comprising methylation of N-demethyl olanzapine with a methylating agent in a solvent comprising dichloromethane, methanol, or a mixture of dichloromethane and methanol.

15. The process according to claim 14, wherein a methylating agent comprises dimethyl sulfate, or a mixture of formaldehyde and formic acid, or methyl iodide.

16. The process according to claim 14, wherein a methylating agent comprises dimethyl sulfate.

17. The process according to claim 14, wherein methylation occurs in the presence of a base.

18. The process according to claim 14, wherein methylation occurs in the presence of a base comprising sodium hydroxide, sodium carbonate, sodium bicarbonate, sodium methoxide, potassium hydroxide, potassium carbonate, potassium bicarbonate, potassium tertiary butoxide, or a mixture of any two or more thereof.

19. The process according to claim 14, wherein methylation occurs in the presence of sodium hydroxide.
